(19) United States
(12) Patent Application Publication
(10) Pub. No.: US 2025/0388756 A1
(43) Pub. Date: Dec. 25, 2025

(54) NOVEL FLUORESCENT COMPOUND, AND LIPID BILAYER DYEING METHOD AND ENDOCYTOSIS DETECTION METHOD USING SAID COMPOUND

(71) Applicant: DOJINDO LABORATORIES, Kamimashiki-gun Kumamoto (JP)

(72) Inventors: Masataka TAKAHASHI, Kamimashiki-gun Kumamoto (JP); Ryo SEINO, Kamimashiki-gun Kumamoto (JP); Takatoshi EZOE, Kamimashiki-gun Kumamoto (JP); Munetaka ISHIYAMA, Kamimashiki-gun Kumamoto (JP)

(21) Appl. No.: 18/023,385

(22) PCT Filed: Aug. 27, 2021
(86) PCT No.: PCT/JP2021/031626

§ 371 Date: Sep. 1, 2025

(30) Foreign Application Priority Data
Aug. 27, 2020 (JP) 2020-143432

(51) Int. Cl. C09B 57/08; C09B 5/62; G01N 33/50; G01N 33/58

(52) U.S. Cl. CPC C09B 57/08; C09B 5/62; G01N 33/5005; G01N 33/582

(57) ABSTRACT

Disclosed are: a fluorescent compound represented by general formula (I), (I)′, or (I)″, as a fluorescent compound which has high retention in a lipid bilayer, has excellent ease of handling, and can achieve uniform dyeing in a dyeing target; and a lipid bilayer dyeing method and an endocytosis detection method using said compound.

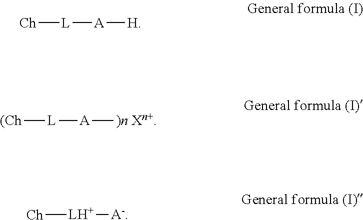

Ch is a hydrophobic-field-sensitive fluorescent chromophore in which the fluorescence intensity increases in hydrophobic environments; A-H is an anionic functional group capable of generating anions by deprotonation; Xⁿ⁺ is a positive ion having biological compatibility; n is 1, 2, or 3; L is a linker that binds to a hydrophobic-field-sensitive fluorescent chromophore and links the fluorescent chromophore and the anionic functional group; and LH⁺ indicates a state wherein the linker, which contains a cationic functional group capable of generating cations by protonation, has generated a cation by protonation.

### TECHNICAL FIELD

The present invention relates to a novel fluorescent compound and method for staining lipid bilayer membrane and method for detecting endocytosis using the same.

### BACKGROUND ART

A lipid bilayer membrane involves a variety of biological processes including separation of the inside from the outside of the cell, uptake of substances into the cell, removal of denatured proteins, degraded intracellular organelles and pathogenic microorganisms, signal transduction pathways, maintenance of homeostasis, suppression of diseases and canceration of cells as a component of cell membrane, exosomes, endosomes, and autophagosomes, etc. In the study on cellular structures and intracellular and extracellular biological processes involving lipid bilayer membrane by fluorescence bioimaging to observe molecular dynamics in living cells and tissues, fluorescent compounds with high molecular specificity, low cytotoxicity, and high retention at the staining site are important. The fluorescent compound used for specific staining of lipid bilayer membrane has a structure in which an atomic group of high lipid affinity such as long alkyl chain is bonded to a fluorescent chromophore, and compounds having structures represented by chemical formulae 1 or 2 shown below have been proposed as the compounds applicable to living cells (see Patent Literature 1 and Patent Literature 2, respectively).

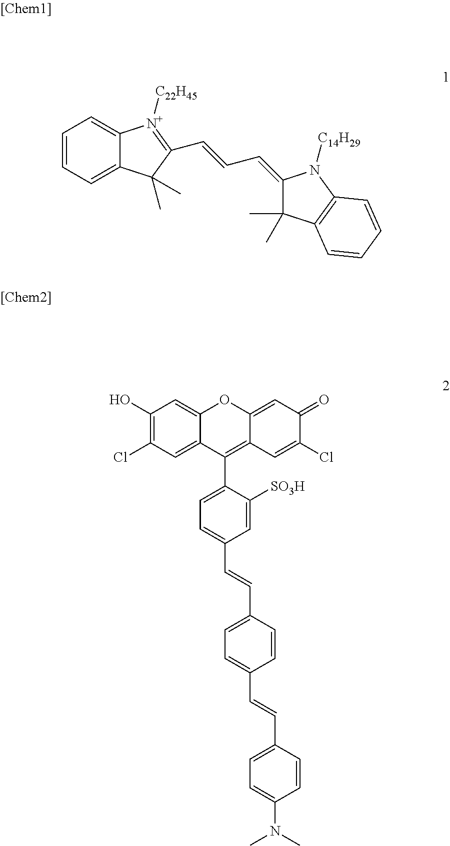

### CITATION LIST

### Patent Literature

Patent Literature 1: U.S. Pat. No. 5,665,328 B1

Patent Literature 2: U.S. Pat. No. 9,651,494 B2

### SUMMARY OF PRESENT INVENTION

### Technical Problem

However, conventional fluorescent compounds such as those represented by the chemical formulae 1 and 2 shown above have a problem that its low retention in lipid bilayer membrane such as cell membrane and migrate to the interior of cells and vesicles after a long time after staining. In addition, these fluorescent compounds require washing after staining, which is a complicated operation. Furthermore, the fluorescent compound represented by the chemical formula 1 shown above has a problem that its low water solubility requires a special diluent, and precipitation and aggregation make it difficult to stain uniformly.

The present invention has been made in view of such circumstances and an object of the present invention is to provide a fluorescent compound that is highly retentive in a lipid bilayer membrane, excellent in operability, and capable of uniformly staining an object to be stained and a method for staining lipid bilayer membrane and method for detecting endocytosis using the same.

### Solution to Problem

The first aspect of the present invention in accordance with the purpose above is to solve the above problem by providing a fluorescent compound represented by general formulae (I), (I)′ or (I)″ shown below.

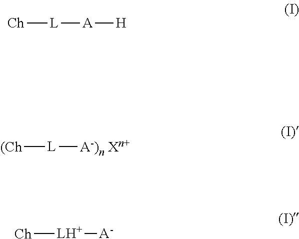

In the general formulae (I), (I)′ and (I)″ shown above,
Ch is a hydrophobic field-sensitive fluorescent chromophore of which fluorescent intensity increases under hydrophobic environment,
A-H is an anionic functional group that can be anionized by deprotonation,
Xⁿ⁺ is a biocompatible cation,
n is 1, 2 or 3,
L is a linker, an atomic group which bonded to carbon or nitrogen atom of the hydrophobic field-sensitive fluorescent chromophore and links the hydrophobic field-sensitive chromophore and the anionic functional group, and
LH⁺ represents a state in which the linker containing a cationic functional group which can be cationized by protonation is protonated and cationized.

In the fluorescent compound according to the first aspect of the present invention, the hydrophobic field-sensitive fluorescent chromophore Ch may be peryleneimide or naphthaleneimide group.

In the fluorescent compound according to the first aspect of the present invention, the anionic functional group A-H may be any one of carboxylic group, sulfuric acid group, sulfonic acid group and phosphoric acid group.

In the fluorescent compound according to the first aspect of the present invention, the linker L may be an atomic group represented by the general formula (II) shown below.

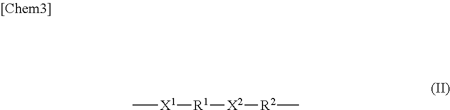

In the general formula (II) shown above, X¹ and X² are independently covalent bonds or atomic groups represented by any one of the following formulae (i) to (iv) shown below, respectively,
R¹ and R² independently represent —(CH₂)ₙ—, —(CH₂CH₂O)ₘ— and —(OCH₂CH₂)ₘ— (m represents a natural number of 1 to 4, and n represents a natural number of 1 to 10, and total carbon number of R¹ and R² is 10 or less.).

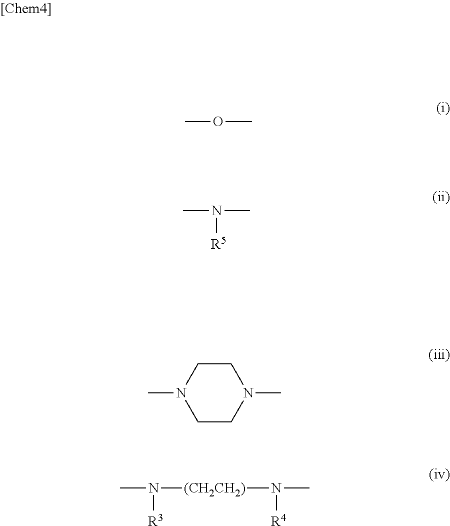

In the formulae (i) to (iv) shown above, R³, R⁴ and R⁵ each dependently represent alkyl groups having the carbon number of 1 to 10.

In the fluorescent compound according to the first aspect of the present invention, the linker L is preferably an atomic group represented by any one of general formulae (III), (IV), (V), (VI), (VII) and (VIII) shown below.

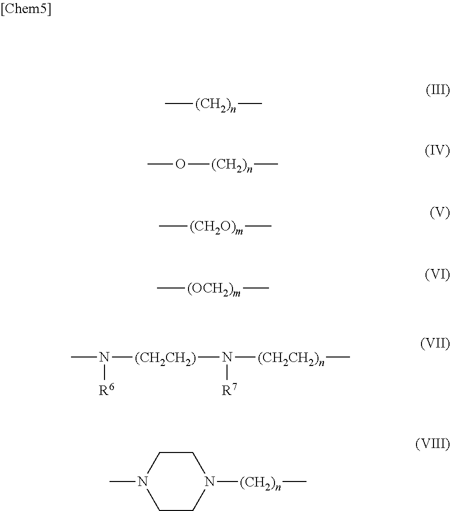

In the general formulae (III), (IV), (V), (VI), (VII) and (VIII) shown above, m represents a natural number of 1 to 4 and n represents a natural number of 1 to 10.

In the fluorescent compound according to the first aspect of the present invention, the linker L may be the atomic group represented by the general formula (VII) or (VIII) shown above.

The fluorescent compound according to the first aspect of the present invention is preferably a compound represented by any one of formulae (1), (2) or (3) shown below or a salt thereof.

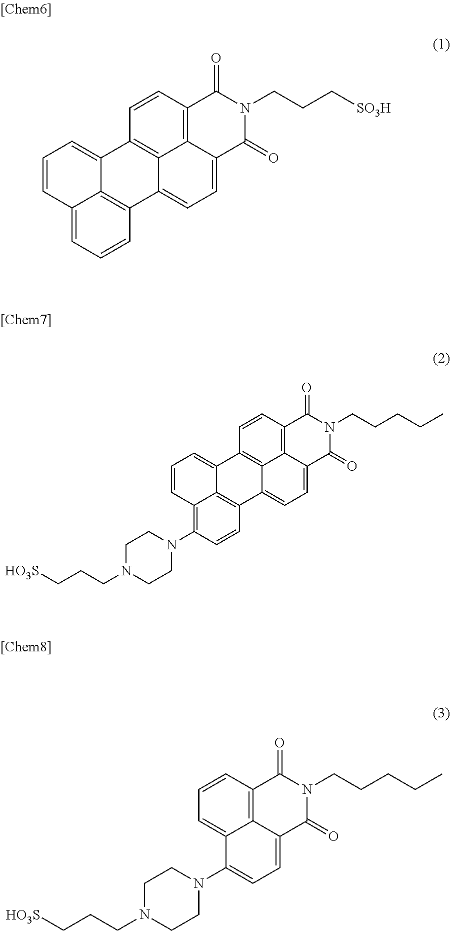 [SMILES: O=C1c2ccc3c4cccc5cccc(c6ccc(c2c36)C(=O)N1CCCS(=O)(=O)O)c54 ; CCCCCN1C(=O)c2ccc3c4cccc5c(N6CCN(CCCS(=O)(=O)O)CC6)ccc(c6ccc(c2c36)C1=O)c54 ; CCCCCN1C(=O)c2cccc3c(N4CCN(CCCS(=O)(=O)O)CC4)ccc(c23)C1=O]

In the fluorescent compound according to the first aspect of the present invention, the biocompatible cation Xⁿ⁺ may be any one of alkali metal ion, alkali earth metal ion and ammonium ion.

The second aspect of the present invention is to solve the above problem by providing a method for staining lipid bilayer membrane comprising a step for providing a sample containing lipid bilayer membrane; and
a step for contacting one or more fluorescent compounds according to the first aspect of the present invention to the lipid bilayer membrane in the sample to stain the lipid bilayer membrane with the fluorescent compound.

The third aspect of the present invention is to solve the above problem by providing a method for detecting endocytosis comprising: a step for providing a sample containing cell;
a step for contacting one or more fluorescent compounds represented by general formulae (I) or (I)′ shown below to the cells in the sample to stain endosome in the cell with the fluorescent compound; and
a step for detecting fluorescence from the stained endosome.

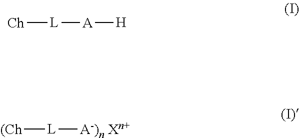

In the general formulae (I), (I)′ and (I)″ shown above,
Ch is a hydrophobic field-sensitive fluorescent chromophore of which fluorescent intensity increases under hydrophobic environment,
A-H is an anionic functional group that can be anionized by deprotonation,
Xⁿ⁺ is a biocompatible cation,
n is 1, 2 or 3,
L is a linker, an atomic group represented by formulae (VII) or (VIII) shown below, which bonded to carbon or nitrogen atom of the hydrophobic field-sensitive fluorescent chromophore and links the hydrophobic field-sensitive chromophore and the anionic functional group.

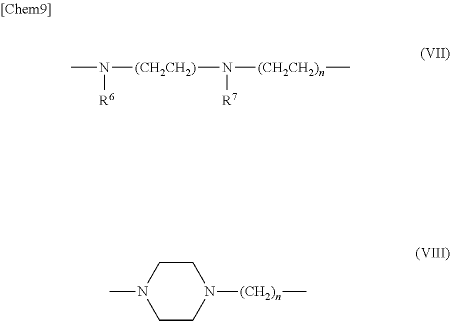

In the method for detecting endocytosis according to the third aspect of the present invention, the hydrophobic field-sensitive fluorescent chromophore Ch of the fluorescent compound may be peryleneimide or naphthaleneimide group.

In the method for detecting endocytosis according to the third aspect of the present invention, the anionic functional group A-H of the fluorescent compound may be any one of carboxyl, sulfate, sulfonic acid, and phosphoric acid groups.

In the method for detecting endocytosis according to the third aspect of the present invention, the fluorescent compound is preferably a compound represented by any one of formulae (2) or (3) shown below or a salt thereof.

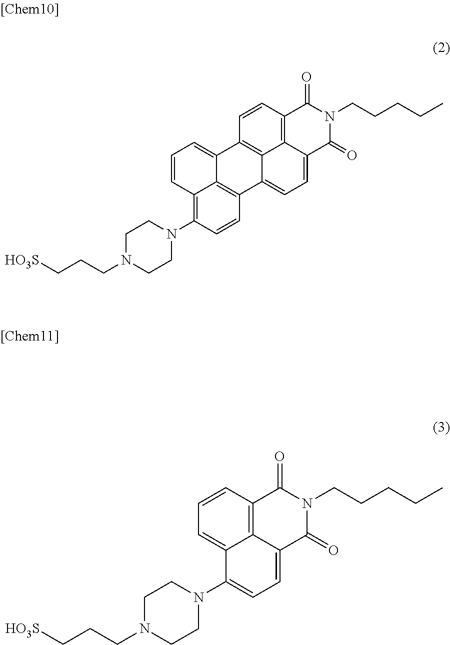 [SMILES: CCCCCN1C(=O)c2ccc3c4cccc5c(N6CCN(CCCS(=O)(=O)O)CC6)ccc(c6ccc(c2c36)C1=O)c54 ; CCCCCN1C(=O)c2cccc3c(N4CCN(CCCS(=O)(=O)O)CC4)ccc(c23)C1=O]

In the method for detecting endocytosis according to the third aspect of the present invention, the biocompatible cation Xⁿ⁺ of the fluorescent compound may be any one of alkali metal ion, alkali earth metal ion and ammonium ion.

### Advantageous Effects of Present invention

The fluorescent compounds of the present invention have high retention in lipid bilayer membrane and can stain lipid bilayer membrane specifically and uniformly for a long time without migration in cells or vesicles. In addition, the fluorescent compounds of the present invention have high solubility in water and do not aggregate or precipitate during solution preparation, which enables uniform staining of lipid bilayer membrane. Furthermore, since the fluorescent compounds of the present invention efficiently migrate into the lipid bilayer membrane, no washing operation is required to remove excess fluorescent compounds, and the staining can be performed by a simple operation. When the linker L has a nitrogen atom with lone pair electrons at a position where it cannot conjugate with the π-electron system of the fluorescent chromophore, as represented by the formula (VII) or (VIII) shown above, pH responsiveness may be provided to fluorescence emission because the quenching of fluorescence due to photoinduced electron transfer can be controlled by pH. Therefore, it is useful for detecting acidic vesicles such as endosomes formed during endocytosis.

The present invention also provides a method for staining lipid bilayer membrane and a method for detecting endocytosis, which can stain lipid bilayer membrane specifically and uniformly with a simple operation.

### BRIEF DESCRIPTION OF DRAWINGS

FIG. 1 represents fluorescence and excitation spectra of Compound (1).

FIG. 2 represents the influence of hydrophobicity of solvents on the emission intensity of Compound (1).

FIG. 3 represents fluorescence and excitation spectra of Compound (2).

FIG. 4 represents the influence of hydrophobicity of solvents on the emission intensity of Compound (2).

FIG. 5 represents fluorescence and excitation spectra of Compound (3).

FIG. 6 represents the influence of hydrophobicity of solvents on the emission intensity of Compound (3).

FIG. 7 represents the influence of pH of solvents on the emission intensity of Compound (3).

FIG. 8 represents a confocal laser micrograph showing the results of a staining test of HeLa cells.

FIG. 9 represents a confocal laser micrograph showing the results of a staining test of endosomes.

### DESCRIPTION OF EMBODIMENTS

A fluorescent compound according to first embodiment of the present invention (hereinafter it may be abbreviated to “fluorescent compound”.) is represented by general formulae (I), (I)′ or (I)′ shown below.

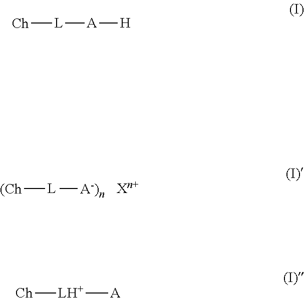

In the general formulae (I), (I)′ and (I)″ shown above,
Ch is a hydrophobic field-sensitive fluorescent chromophore of which fluorescent intensity increases under hydrophobic environment,
A-H is an anionic functional group that can be anionized by deprotonation,
Xⁿ⁺ is a biocompatible cation,
n is 1, 2 or 3,
L is a linker, an atomic group which bonded to carbon or nitrogen atom of the hydrophobic field-sensitive fluorescent chromophore and links the hydrophobic field-sensitive chromophore and the anionic functional group, and

LH⁺ represents a state in which the linker containing a cationic functional group which can be cationized by protonation is protonated and cationized.

Hereinafter, the fluorescent compound and each component of the compound (Ch, A-H, Xⁿ⁺, and L) will be illustrated more specifically with preferred embodiments.

### Hydrophobic Field-Sensitive Fluorescent Chromophore Ch

The term “hydrophobic field-sensitive fluorescent chromophore” in the present disclosure broadly refers to a fluorescent chromophore in which one or both of the emission intensity and wavelength change between hydrophobic and hydrophilic environments, and for the purposes of the present invention, more preferably a fluorescent chromophore of which emission intensity increases under a hydrophobic environment than under a hydrophilic environment. The increase in fluorescence intensity of a fluorescent chromophore in a hydrophobic environment compared to a hydrophilic environment enables the fluorescent compound to emit strong fluorescence when the fluorescent compound is in the lipid bilayer membrane, a hydrophobic environment, thereby enabling specific staining of lipid bilayer membrane. Specific examples of fluorescent chromophores of which fluorescent intensity increases under hydrophobic environment include DAPI (4′,6-diamidino-2-phenylindole), ANS (8-anilinonaphthalene-1-sulfonic acid), peryleneimide or naphthaleneimide groups, etc., preferably peryleneimide or naphthaleneimide groups.

### Anionic Functional Group A-H

Anionic functional group A-H is a functional group that can deprotonate to produce anions in, for example, in vivo environment. Lipid bilayer membrane such as cell membrane contain phospholipids such as phosphatidylcholine that have ammonium groups which is cationized in the in vivo environment. If the fluorescent compound has an anionic functional group, the electrostatic interaction between the positive charge of the phospholipid and the negative charge of the anionic functional group may improve the retention of the fluorescent compound in the lipid bilayer membrane.

Specific examples of preferred anionic functional group include carboxylic group (—COOH), sulfuric acid group (—O—SO₃H), sulfonic acid group (—SO₃H), phosphoric acid group (—O—PO(OH)₂) and diphosphoric acid group (—O—PO(OH)—O—PO(OH)₂) and sulfonic acid group is particularly preferred.

### Linker L

Linker L is an atomic group which bonded to carbon or nitrogen atom of the hydrophobic field-sensitive fluorescent chromophore and links the hydrophobic field-sensitive chromophore Ch and the anionic functional group A-H. The length of the linker is not limited as long as the fluorescent chromophore Ch present in the lipid bilayer membrane does not protrude from the lipid bilayer membrane upon electrostatic interaction between the negative charge of the anion generated by deprotonation of the anionic functional group A-H and the positive charge of the phospholipid in the lipid bilayer membrane. The linker L is an atomic group, for example, represented by general formula (II) shown below.

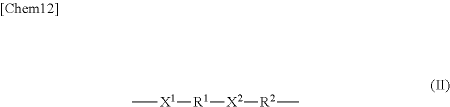

In the general formula (II) shown above, X¹ and X² are independently covalent bonds or atomic groups represented by any one of the following formulae (i) to (iv), respectively,
R¹ and R² each independently represent —(CH₂)ₙ—, —(CH₂CH₂O)ₘ— and —(OCH₂CH₂)ₘ— (m represents a natural number of 1 to 4, and n represents a natural number of 1 to 10, and total carbon number of R¹ and R² is 10 or less.).

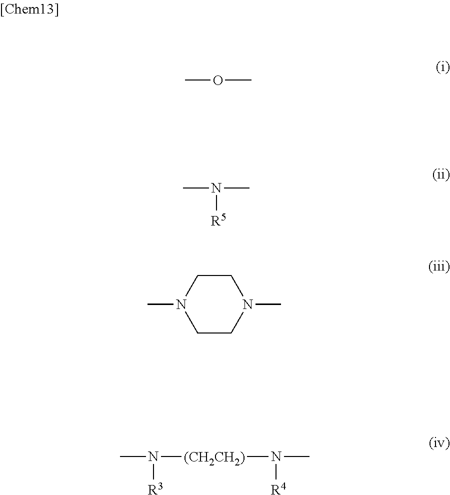

In the formulae (i) to (iv) shown above, R³, R⁴ and R⁵ each independently represent alkyl groups having the carbon number of 1 to 10.

Examples of preferred linker L include an atomic group represented by any one of general formulae (III), (IV), (V), (VI), (VII) and (VIII) shown below.

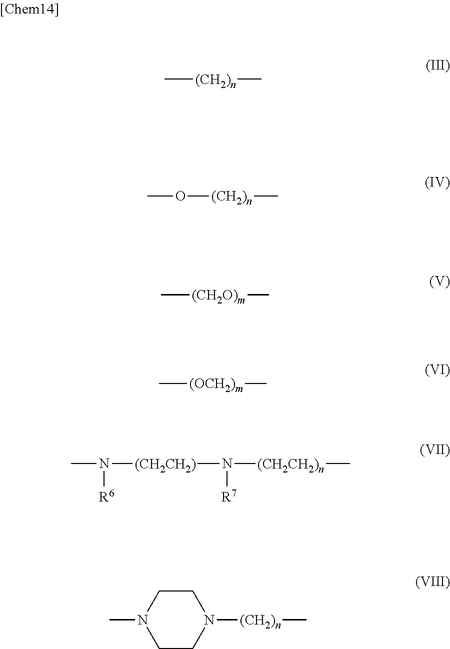

In the general formulae (III), (IV), (V), (VI), (VII) and (VIII) shown above, m represents a natural number of 1 to 4 and n represents a natural number of 1 to 10.

When the linker L is the atomic group represented by the formula (VII) or (VIII) shown above, since it has a nitrogen atom with a lone pair electrons at a position where it cannot conjugate with the π-electron system of the fluorescent chromophore, under neutral or basic conditions where the nitrogen atom is not protonated, the fluorescence from the fluorescent chromophore Ch is quenched by photoinduced electron transfer from the lone pair electrons on the nitrogen atom to the fluorescent chromophore Ch. On the other hand, under acidic conditions where nitrogen atoms is protonated, quenching of fluorescent chromophores by photoinduced electron transfer does not occur. Therefore, when the linker L is an atomic group represented by the formula (VII) or (VIII) shown above, the emission intensity of the fluorescent chromophore can be controlled by pH, thus making it possible to provide pH responsiveness to fluorescent emission (increase the emission intensity under acidic conditions). Therefore, it is useful for detection of acidic vesicles such as endosomes formed during endocytosis.

Specific example of preferred fluorescent compound is the compound represented by the formulae (1), (2) or (3) shown below or the salt thereof.

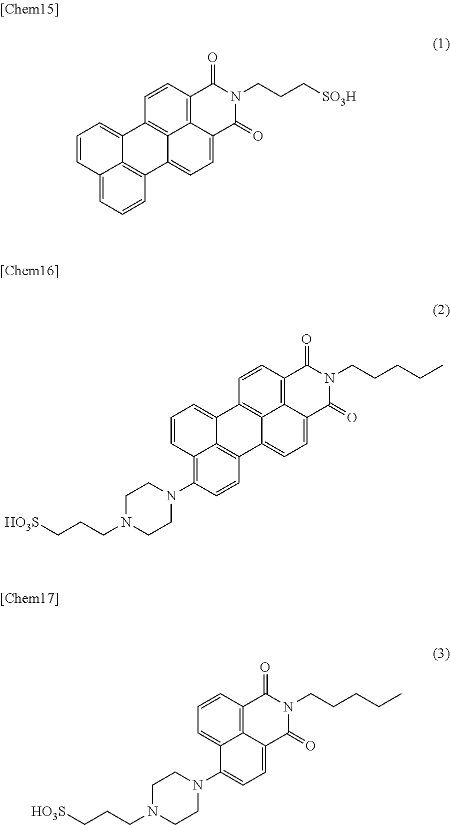 [SMILES: O=C1c2ccc3c4cccc5cccc(c6ccc(c2c36)C(=O)N1CCCS(=O)(=O)O)c54 ; CCCCCN1C(=O)c2ccc3c4cccc5c(N6CCN(CCCS(=O)(=O)O)CC6)ccc(c6ccc(c2c36)C1=O)c54 ; CCCCCN1C(=O)c2cccc3c(N4CCN(CCCS(=O)(=O)O)CC4)ccc(c23)C1=O]

### Biocompatible Cation Xⁿ⁺

When the anionic functional group of the fluorescent compound is deprotonated, the fluorescent compound forms a salt with a cation as a counterion, as represented by general formula (I)′, or the linker containing a cationic functional group that can be cationized by protonation is protonated and cationized to form an intramolecular salt, as represented by general formula (I)″. The cation that serves as the counterion of the deprotonated anionic functional group may be any cation as long as it is biocompatible. The valence n of the biocompatible cation Xⁿ⁺ is 1, 2 or 3. Specific examples of biocompatible cations Xⁿ⁺ include alkali metal ions such as sodium and potassium, alkaline earth metal ions such as magnesium, calcium, and strontium, and ammonium ions.

Fluorescent compounds may be synthesized using any method known in the art. For example, compounds represented by the formulae (1), (2), or (3) shown above may be synthesized according to the scheme shown in the Example below.

A method for staining lipid bilayer membrane according to second embodiment of the present invention comprises a step for providing a sample containing lipid bilayer membrane; and a step for contacting one or more fluorescent compounds according to the first embodiment of the present invention to the lipid bilayer membrane in the sample to stain the lipid bilayer membrane with the fluorescent compound.

The lipid bilayer membrane to be stained includes cell membrane, lipid bilayer membrane constituting intracellular organelles, extracellular vesicles such as endodomes, intracellular vesicles such as exosomes and autophagosomes, viral envelopes.

The preparation of a sample containing lipid bilayer membrane includes, for example, the step for collecting a biological sample containing cells, etc. to be stained or culturing cells to be stained in a culture medium or solid medium, and may include, if necessary, any known isolation operation or pretreatment, such as ultrafiltration, centrifugation, etc.

Although a predetermined amount of the fluorescent compound may be added directly to the sample solution, it is preferable to prepare a solution of a predetermined concentration of the fluorescent compound in advance to control the concentration, etc. Any solvents, buffers, etc. known in the art may be selected and used in the preparation of the solution as appropriate as long as the desired concentration of the solution may be afforded.

Staining of lipid bilayer membrane with the fluorescent compound may be performed, for example, by adding the fluorescent compound to the sample and allowing it to stand for a predetermined period (for example, 5 to 10 minutes). The staining state may be observed using any equipment and methods known in the art, such as fluorescence microscopy (for example, confocal laser microscopy).

A method for detecting endocytosis according to third embodiment of the present invention comprises a step for providing a sample containing cell, a step for contacting one or more fluorescent compounds represented by the formulae (I) or (I)′ shown below to the cells in the sample to stain endosome in the cell with the fluorescent compound, and a step for detecting fluorescence from the stained endosome.

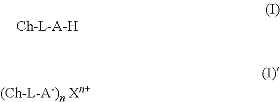

In the general formulae (I), (I)′ and (I)″ shown above,
Ch is a hydrophobic field-sensitive fluorescent chromophore of which fluorescent intensity increases under hydrophobic environment,
A-H is an anionic functional group that can be anionized by deprotonation,
Xⁿ⁺ is a biocompatible cation,
n is 1, 2 or 3,
L is a linker, an atomic group represented by formulae (VII) or (VIII) shown below, which bonded to carbon or nitrogen atom of the hydrophobic field-sensitive fluorescent chromophore and links the hydrophobic field-sensitive chromophore and the anionic functional group.

A detailed description of the fluorescent compound and each component of the compound (Ch, A-H, Xⁿ⁺, and L) is omitted since they are similar to the fluorescent compound according to the first embodiment of the present invention, except that the linker L is represented by the formula (VII) or (VIII) shown below. The cells to be detected may be any cells as long as they undergo endocytosis. The detailed description of the step for staining endosomes in cells is omitted since it is similar to the step for staining lipid bilayer membrane in the method for staining lipid bilayer membrane according to the second embodiment of the present invention. Furthermore, the detailed description of the step for detecting fluorescence from stained endosomes is omitted since it the same as the step for observing the staining state in the description of the staining method for lipid bilayer membrane according the second embodiment of the present invention.

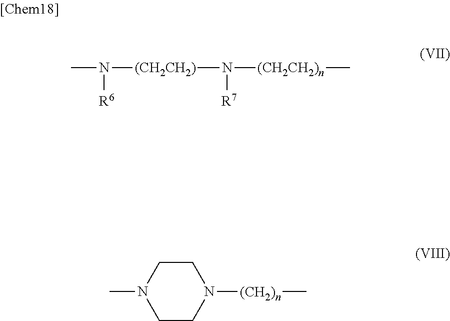

In endocytosis, endosomes that have separated from the cell membrane are kept in a weakly acidic state inside by the action of a proton pump, but when they fuse with lysosomes in the later stages of endocytosis, the internal pH is further decreased. Therefore, for staining endosomes, it is preferable to use a pH-responsive fluorescent compound of which fluorescence intensity increases under acidic conditions. A specific example of such a fluorescent compound includes the compound represented by formula (3) shown below. In conventional staining of endosomes, when low molecular weight fluorescent compound is used, only the contents of the endosome can be stained, making it difficult to examine the membrane structure of the endosome in detail. Although a method of staining endosomal membranes using cDNA encoding a fused protein of a fluorescent protein and a protein expressed organelle-specifically or structure-specifically is known in the art, the compound represented by formula (3) shown below provides a more convenient and inexpensive method of staining endosomes.

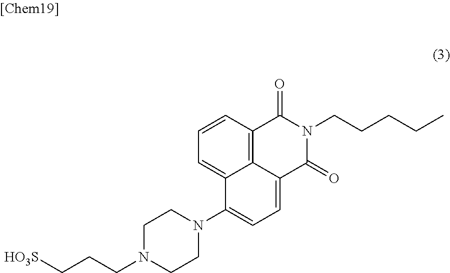 [SMILES: CCCCCN1C(=O)c2cccc3c(N4CCN(CCCS(=O)(=O)O)CC4)ccc(c23)C1=O]

### EXAMPLES

Hereinafter, examples conducted to confirm the effects of the invention will be described.

### Example 1: Synthesis of Fluorescent Compounds

Fluorescent compounds represented by formulae (1), (2), and (3) (hereinafter they may be abbreviated to “Compound (1)”, “Compound (2)” and “Compound (3)”) were synthesized in accordance with the following scheme, respectively. At least a part of the sulfonic acid groups of Compound (1), (2), and (3) may be in the form of salts.

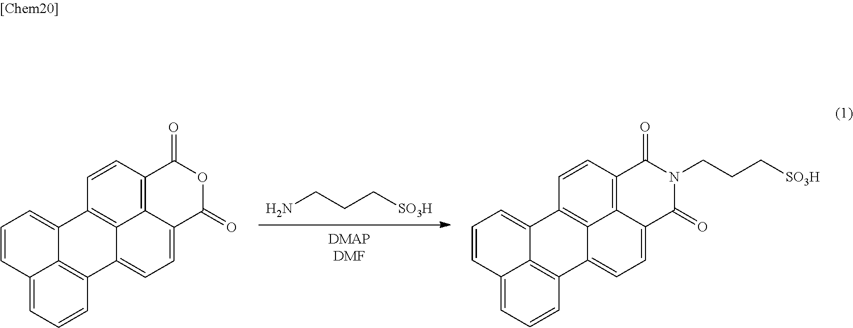 [SMILES: O=C1OC(=O)c2ccc3c4cccc5cccc(c6ccc1c2c63)c54 ; NCCCS(=O)(=O)O ; O=C1c2ccc3c4cccc5cccc(c6ccc(c2c36)C(=O)N1CCCS(=O)(=O)O)c54]

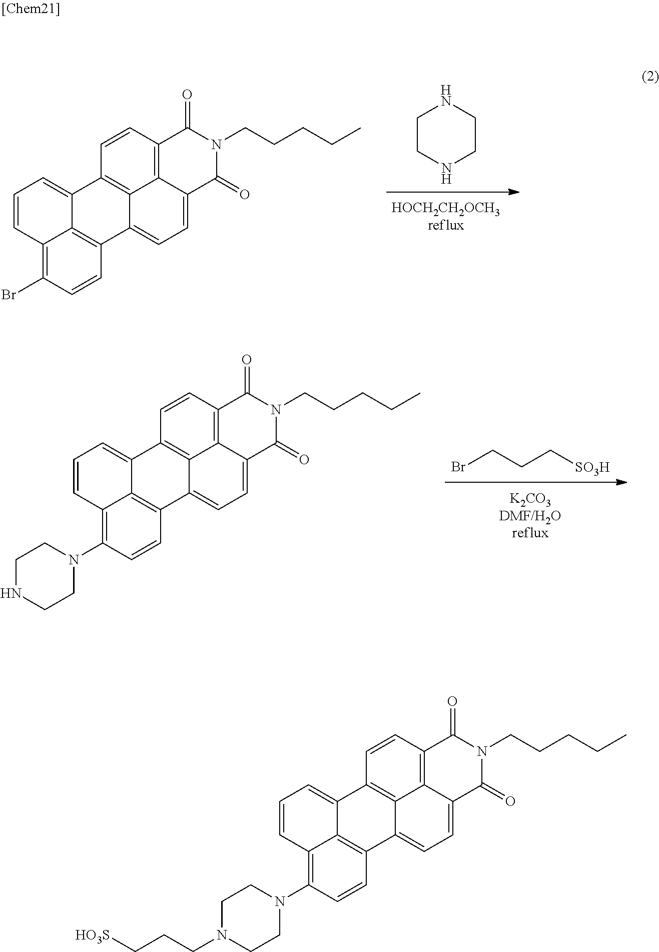 [SMILES: CCCCCN1C(=O)c2ccc3c4cccc5c(Br)ccc(c6ccc(c2c36)C1=O)c54 ; C1CNCCN1 ; CCCCCN1C(=O)c2ccc3c4cccc5c(N6CCNCC6)ccc(c6ccc(c2c36)C1=O)c54 ; O=S(=O)(O)CCCBr ; CCCCCN1C(=O)c2ccc3c4cccc5c(N6CCN(CCCS(=O)(=O)O)CC6)ccc(c6ccc(c2c36)C1=O)c54]

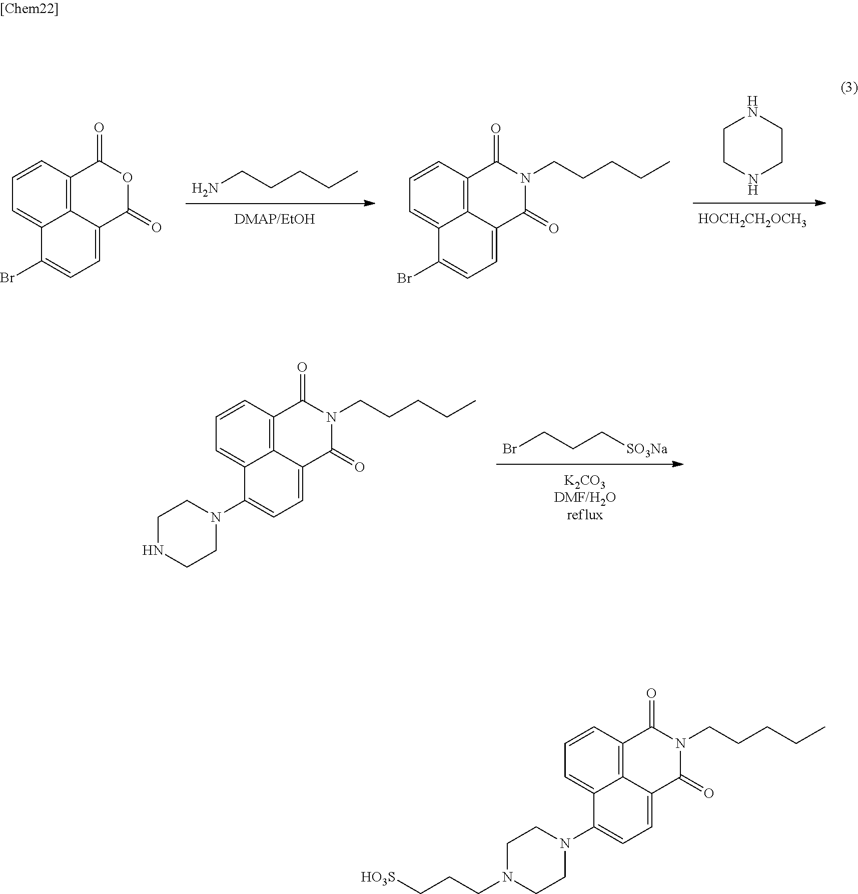

### Example 2: Evaluation of Fluorescence Properties of Fluorescent Compounds

### [1] Measurement of Fluorescence and Excitation Spectra

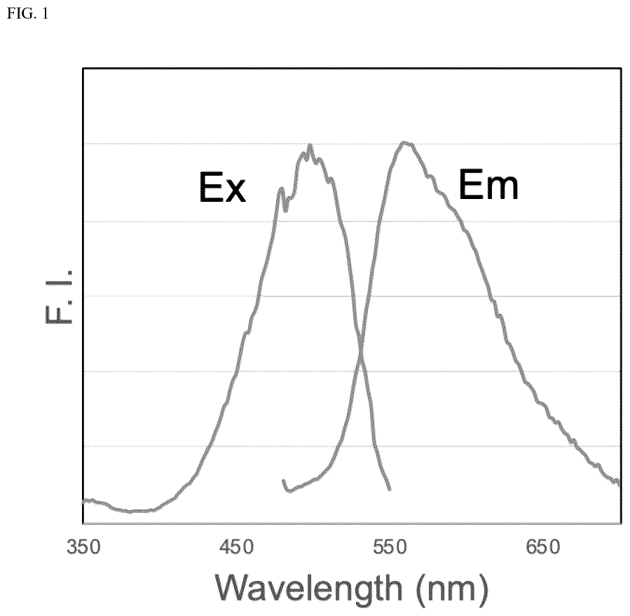

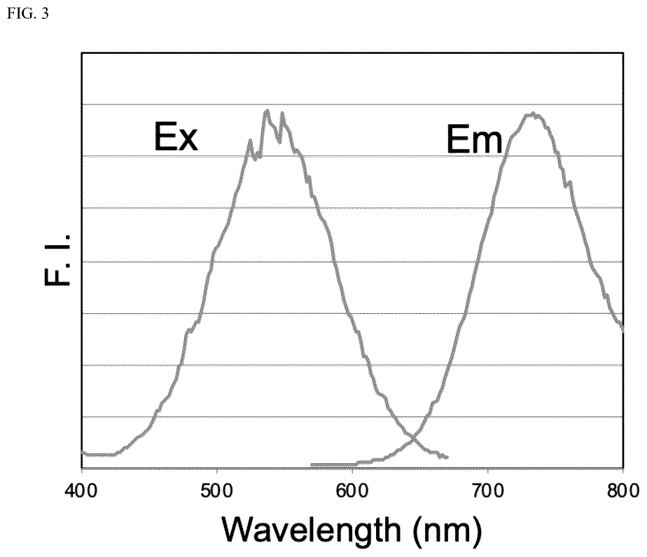

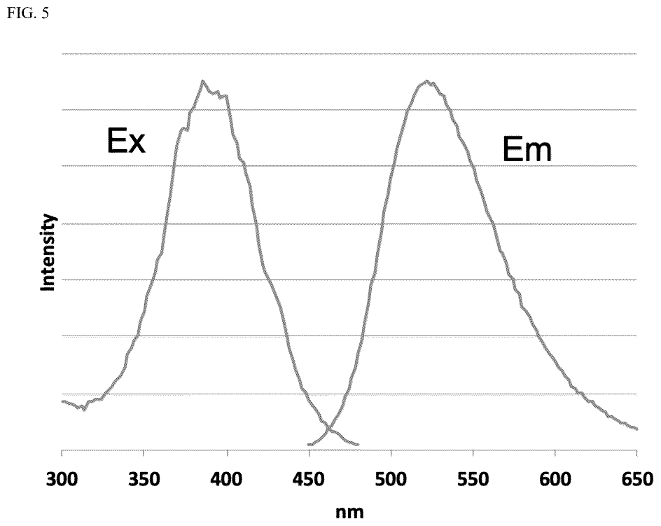

Fluorescence and excitation spectra of Compound (1), (2), and (3) were measured using 1 μmol/L solutions of each compound. The solvents used were DMSO for Compound (1) and (2) and phosphate buffered saline (PBS) for Compound (3). The results for Compound (1), (2), and (3) are shown in FIGS. 1, 3, and 5, respectively. The excitation and measurement wavelengths for each compound in the following experiments were determined based on the wavelengths corresponding to the maximum values of the fluorescence and excitation spectra obtained.

### [2] Investigation of the Effect of Hydrophobicity on Fluorescence Intensity

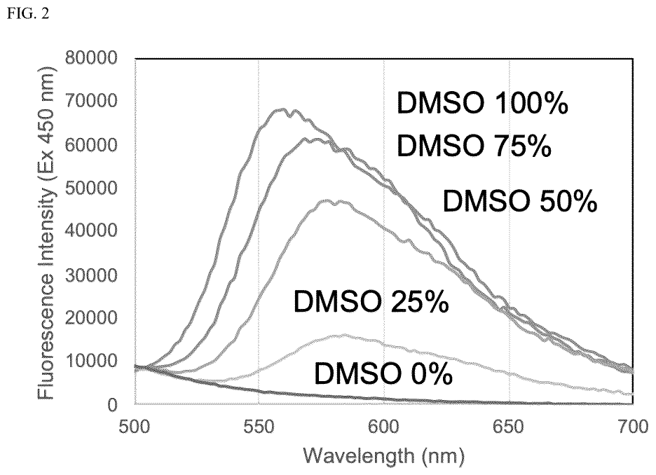

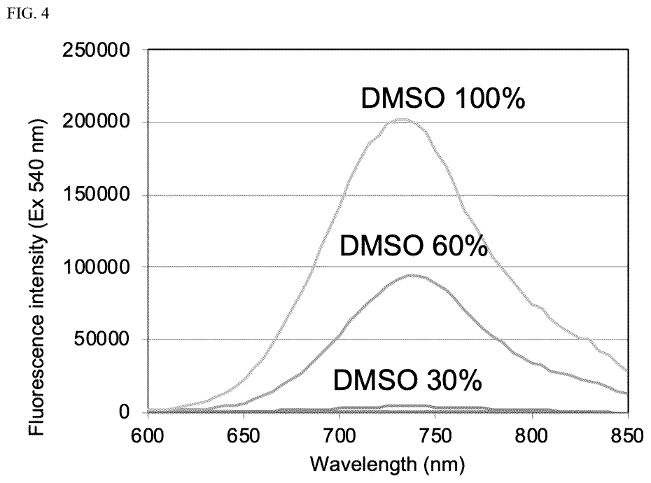

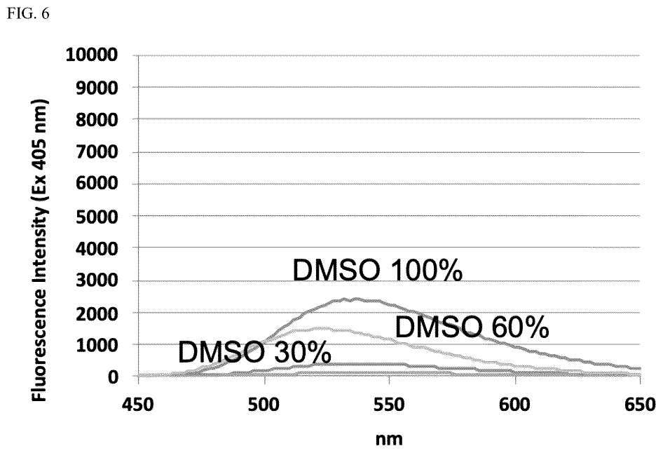

Each compound was dissolved in a mixed solvent of DMSO and PBS with different DMSO concentrations (DMSO concentration: 0%, 25%, 50%, 75%, and 100% for compound (1); DMSO concentration: 0%, 30%, 60%, and 100% for Compound (2) and (3)) so that the final concentration was 1 μmol/L, and the resulting solutions were used to measure fluorescence spectra to investigate the effect of hydrophobicity (DMSO concentration) on fluorescence intensity. The results for Compound (1), (2), and (3) are shown in FIGS. 2, 4, and 6, respectively. For all compounds, an increase in fluorescence intensity with increasing hydrophobicity of the solution was observed.

### [3] Investigation of the Effect of pH on Fluorescence Intensity in Compound (3)

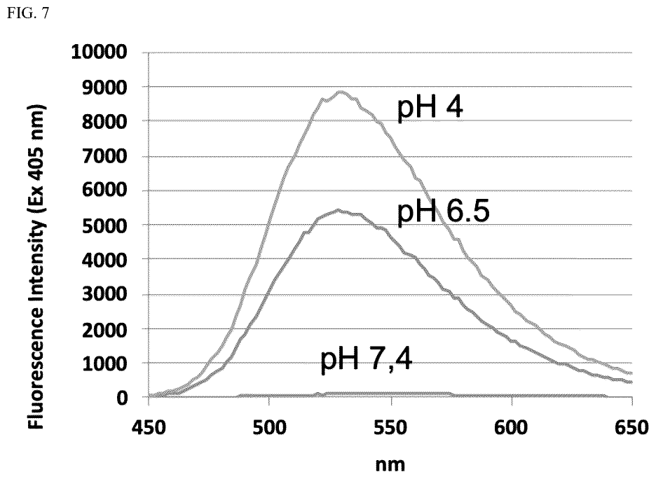

Compound (3) was added to a 1:1 mixture of PBS (pH 7.4), Tris-HCl buffer (pH 6.5), phthalate buffer (pH 4) and DMSO to a final concentration of 1 μmol/L and the fluorescence spectrum was measured. The results are shown in FIG. 7. In Compound (3), a marked increase in fluorescence intensity was observed with decreasing pH. This means that under neutral or basic conditions where the nitrogen atom which is not conjugated with the π-electron system of the naphthaleneimide group, the fluorescent chromophore on the piperazine ring, is not protonated, the fluorescence is quenched by photoinduced electron transfer from the lone pair electrons on the nitrogen atom to the naphthaleneimide group, whereas under acidic conditions, where the nitrogen atom is protonated, quenching of the naphthaleneimide group by photoinduced electron transfer does not take place.

### Example 3: Cell Staining Test

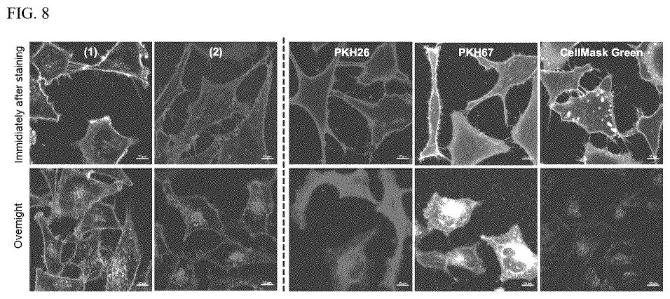

HeLa cells were seeded onto μ-slide 8 well plates (ibidi) and cultured overnight in an incubator (37° C., 5% CO₂, MEM medium (containing 10% fetal bovine serum and 1% penicillin/streptomycin)). The medium was removed, and each fluorescent compound (Compound (1), (2) and commercially available fluorescent compounds (PKH26, PKH67, CellMask Green (Thermofisher) as control) diluted in MEM medium was added and incubated at 37° C. for 5 minutes. Supernatant was removed, replaced with MEM medium and observed with a confocal laser microscope. The results are shown in FIG. 8. In the case of all fluorescent compounds, the cell membrane was specifically stained immediately after staining, but after leaving overnight, it was observed that the fluorescent compounds remained in the cell membrane in the case of Compound (1) and (2), but for the other fluorescent compounds, migration into the cytoplasmic interior or leakage from the cell was observed. These results confirm that Compound (1) and (2) exhibit higher retention in lipid bilayer membrane than conventional fluorescent compounds.

### Example 4: Endosome Staining Test

HeLa cells were seeded on μ-slide 8 well plates (ibidi) and cultured overnight in an incubator (37° C., 5% CO₂ presence, MEM medium (containing 10% fetal bovine serum and 1% penicillin/streptomycin)). The medium was removed and wortmannin (100 nM: a PI-3 kinase inhibitor that hypertrophies early endosomes) diluted in MEM medium Various fluorescent compounds (Compound (3), pHrodo Dex (Thermofisher), FM1-43 (Molecular Probe)) diluted with MEM medium were added and incubated for 30 minutes at 37° C. The supernatant was removed, and the cells were incubated at 37° C. for 30 minutes. The supernatant was removed, replaced with MEM medium and observed with a confocal laser microscope. In addition, instead of adding fluorescent compounds, the same experiment was performed using HeLa cells expressing Rab5-RFP (cDNA encoding a fusion protein in which a fluorescent protein is fused to an endosomal membrane-specific protein) before addition of wortmannin. The same experiments were also performed on the control group (CTRL) without adding wortmannin.

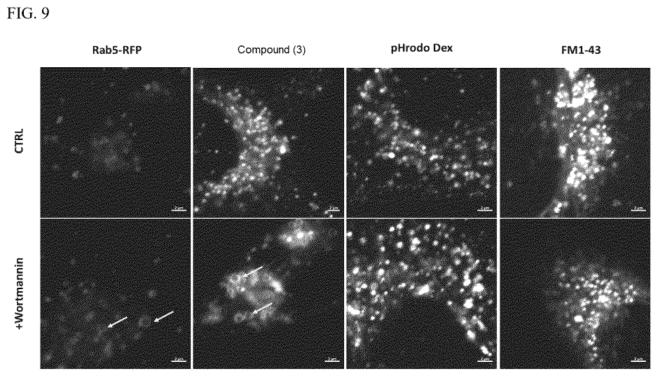

The results are shown in FIG. 9. When Rab5-RFP was expressed in the cells and when the cells were stained using Compound (3), it was confirmed that only the endosomal membrane was specifically stained by enlarging the endosomes by adding wortmannin, while for the other fluorescent compounds for the other fluorescent compounds, the contents of the endosomes were stained, and no specific staining image of the endosomal membrane was observed.

Various embodiments and variations of the present invention are possible without departing from the broad spirit and scope of the present invention. The embodiments described above are for the purpose of illustrating the present invention and do not limit the scope of the present invention. In other words, the scope of the present invention is indicated by the claims, not the embodiments. And various variations made within the scope of the claims and within the equivalent meaning of the present invention are considered within the scope of the present invention.

The present application is based on Japanese Patent Application No. 2020-143432, filed on Aug. 27, 2020, including its specification, claims, drawings, and abstract. The disclosure in the above Japanese patent application is incorporated herein by reference in its entirety.

1. A fluorescent compound represented by general formulae (I), (I)′ or (I)″ shown below. 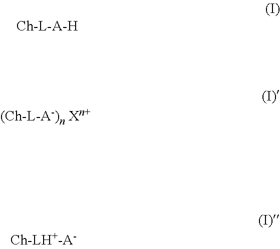
In the general formulae (I), (I)′ and (I)″ shown above,
Ch is a hydrophobic field-sensitive fluorescent chromophore of which fluorescent intensity increases under hydrophobic environment,
A-H is an anionic functional group that can be anionized by deprotonation,
Xⁿ⁺ is a biocompatible cation,
n is 1, 2 or 3,
L is a linker, an atomic group which bonded to carbon or nitrogen atom of the hydrophobic field-sensitive fluorescent chromophore and links the hydrophobic field-sensitive chromophore and the anionic functional group, and
LH⁺ represents a state in which the linker containing a cationic functional group which can be cationized by protonation is protonated and cationized.

2. The fluorescent compound according to claim 1, wherein the hydrophobic field-sensitive fluorescent chromophore Ch is peryleneimide or naphthaleneimide group.

3. The fluorescent compound according to claim 1, wherein the anionic group A-H is any one of carboxylic group, sulfuric acid group, sulfonic acid group and phosphoric acid group.

4. The fluorescent compound according to claim 1, wherein the linker L is an atomic group represented by the following general formula (II). 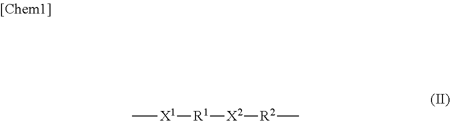
In the general formula (II) shown above, X¹ and X² are independently covalent bonds or atomic groups represented by any one of the following formulae (i) to (iv), respectively,
R¹ and R² each independently represent —(CH₂)ₙ—, —(CH₂CH₂O)ₘ— and (OCH₂CH₂)ₘ— (m represents a natural number of 1 to 4, and n represents a natural number of 1 to 10, and total carbon number of R¹ and R² is 10 or less.). 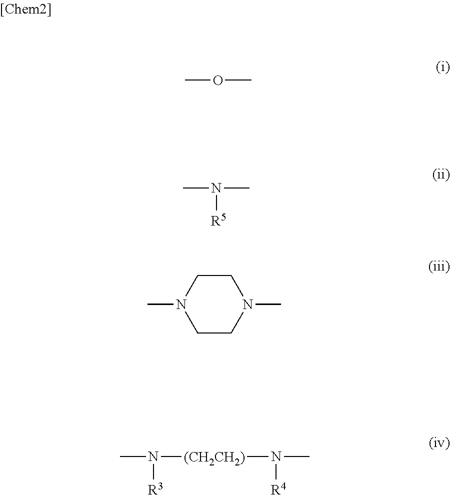
In the formulae (i) to (iv) shown above, R³, R⁴ and R⁵ each independently represent alkyl groups having the carbon number of 1 to 10.

5. The fluorescent compound according to claim 4, wherein the linker L is an atomic group represented by any one of general formulae (III), (IV), (V), (VI), (VII) and (VIII) shown below. 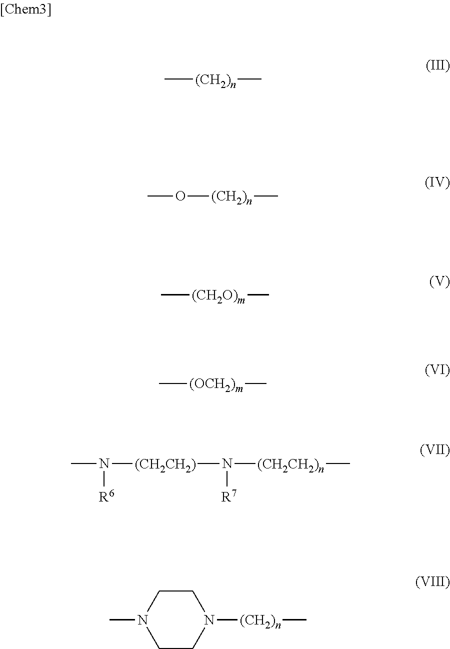
In the general formulae (III), (IV), (V), (VI), (VII) and (VIII) shown below, m represents a natural number of 1 to 4 and n represents a natural number of 1 to 10.

6. The fluorescent compound according to claim 5, wherein the linker L is the atomic group represented by the general formula (VII) or (VIII) shown above.

7. The fluorescent compound according to claim 1, wherein the compound is a compound represented by any one of formulae (1), (2) or (3) or a salt thereof.

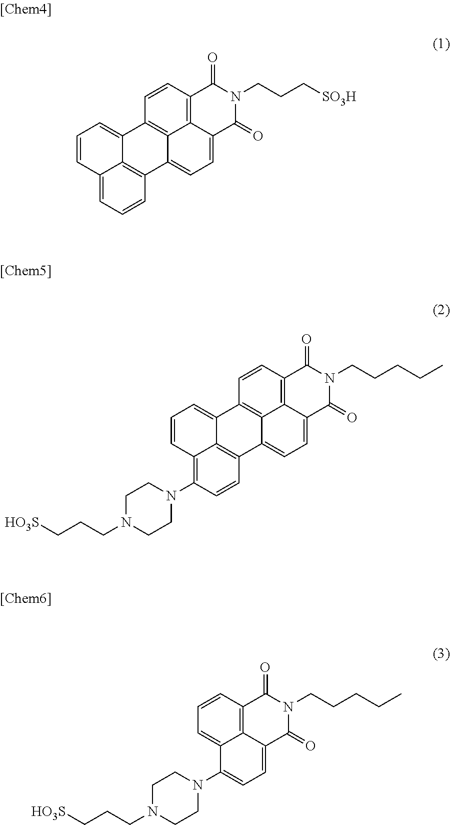 [SMILES: O=C1c2ccc3c4cccc5cccc(c6ccc(c2c36)C(=O)N1CCCS(=O)(=O)O)c54 ; CCCCCN1C(=O)c2ccc3c4cccc5c(N6CCN(CCCS(=O)(=O)O)CC6)ccc(c6ccc(c2c36)C1=O)c54 ; CCCCCN1C(=O)c2cccc3c(N4CCN(CCCS(=O)(=O)O)CC4)ccc(c23)C1=O]

8. The fluorescent compound according to claim 1, wherein the biocompatible cation Xⁿ⁺ is any one of alkali metal ion, alkali earth metal ion and ammonium ion.

9. A method for staining lipid bilayer membrane comprising:
a step for providing a sample containing lipid bilayer membrane; and
a step for contacting one or more fluorescent compounds according to claim 1 to the lipid bilayer membrane in the sample to stain the lipid bilayer membrane with the fluorescent compound.

10. A method for detecting endocytosis comprising:
a step for providing a sample containing cell;
a step for contacting one or more fluorescent compounds represented by formulae (I) or (I)′ to the cells in the sample to stain endosome in the cell with the fluorescent compound; and
a step for detecting fluorescence from the stained endosome. 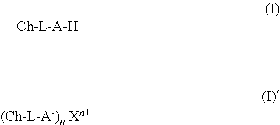
In the general formulae (I), (I)′ and (I)″ shown above,
Ch is a hydrophobic field-sensitive fluorescent chromophore of which fluorescent intensity increases under hydrophobic environment,
A-H is an anionic functional group that can be anionized by deprotonation,
Xⁿ⁺ is a biocompatible cation,
n is 1, 2 or 3,
L is a linker, an atomic group represented by formulae (VII) or (VIII) shown below, which bonded to carbon or nitrogen atom of the hydrophobic field-sensitive fluorescent chromophore and links the hydrophobic field-sensitive chromophore and the anionic functional group.

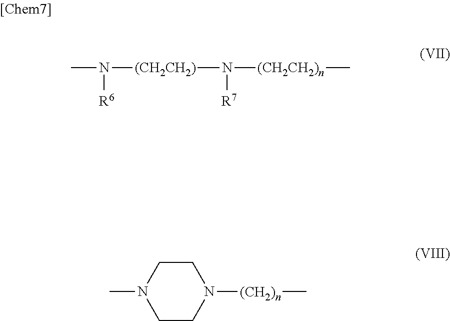

11. The method for detecting endocytosis according to claim 10, wherein the hydrophobic field-sensitive fluorescent chromophore Ch of the fluorescent compound is peryleneimide or naphthaleneimide group.

12. The method for detecting endocytosis according to claim 10, wherein the anionic group A-H of the fluorescent compound is any one of carboxylic group, sulfuric acid group, sulfonic acid group and phosphoric acid group.

13. The method for detecting endocytosis according to claim 10, wherein the fluorescent compound is represented by any one of formulae (1), (2) or (3) or a salt thereof.

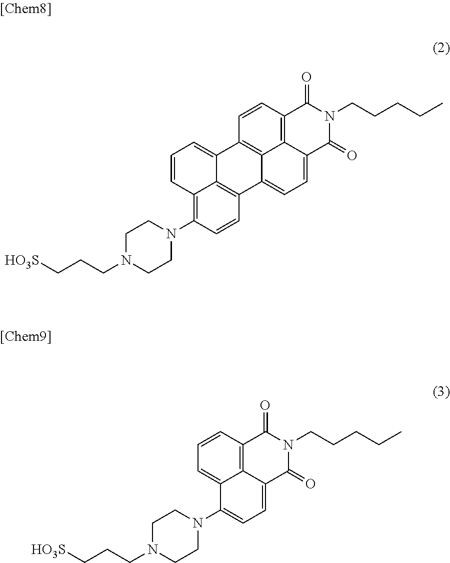 [SMILES: CCCCCN1C(=O)c2ccc3c4cccc5c(N6CCN(CCCS(=O)(=O)O)CC6)ccc(c6ccc(c2c36)C1=O)c54 ; CCCCCN1C(=O)c2cccc3c(N4CCN(CCCS(=O)(=O)O)CC4)ccc(c23)C1=O]

14. The method for detecting endocytosis according to claim 10, wherein the biocompatible cation Xⁿ⁺ of the fluorescent any one of alkali metal ion, alkali earth metal ion and ammonium ion.
